(19) United States
(12) Patent Application Publication
(10) Pub. No.: US 2018/0344517 A1
(43) Pub. Date: Dec. 6, 2018

(54) METHODS AND APPARATUSES FOR THE THERMAL TREATMENT OF NEUROLOGIC AND PSYCHIATRIC DISORDERS

(71) Applicant: Eric A. NOFZINGER, Allison Park, PA (US)

(72) Inventor: Eric A. NOFZINGER, Allison Park, PA (US)

(21) Appl. No.: 16/001,873

(22) Filed: Jun. 6, 2018

Related U.S. Application Data
(60) Provisional application No. 62/515,992, filed on Jun. 6, 2017.

(51) Int. Cl. A61F 7/10; A61B 5/024; A61B 5/00

(52) U.S. Cl. CPC A61F 7/10; A61B 5/02405; A61F 2007/0003; A61B 5/4836; A61B 5/6803

(57) ABSTRACT

Method and apparatuses for applying region cooling to modulate the autonomic nervous system (and particularly the parasympathetic nervous systems) to treat a medical disorder. Described herein are methods and apparatuses for modulating a patient's parasympathetic nervous system by simulating a diving reflex using localized cooling.

### CROSS REFERENCE TO RELATED APPLICATIONS

This patent application claims priority to U.S. Provisional Patent Application No. 62/515,992, filed on Jun. 6, 2017, titled “FOREHEAD COOLING DEVICE TO STIMULATE THE PARASYMPATHETIC NERVOUS SYSTEM FOR THE TREATMENT OF NEUROLOGIC, PSYCHIATRIC OR CARDIAC DISORDERS.”

This application may be related to one or more of: U.S. application Ser. No. 14/938,705, filed Nov. 11, 2015 (and its priority documents); U.S. application Ser. No. 15/921,528, filed Mar. 14, 2018 (and its priority documents); U.S. application Ser. No. 14/758,438, filed Jan. 2, 2014 (and its priority documents); U.S. application Ser. No. 15/521,375, filed Jan. 27, 2016 (and its priority documents); U.S. application Ser. No. 15/597,057, filed May 16, 2017 (and its priority documents); and U.S. application Ser. No. 15/597,078, filed May 16, 2017 (and its priority documents). Each of these patent applications is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are devices and methods for stimulating the parasympathetic nervous system for the treatment of neurologic and/or psychiatric disorders. These devices may include a cooling applicator (garment, cap, etc.) for application to the face (e.g., forehead) of a patient and that can be applied to stimulate the parasympathetic nervous system in a medical disorder in which enhancing parasympathetic tone is therapeutic. Embodiments of cooling systems designed for optimal use in humans are also described.

### BACKGROUND

Extensive clinical research supports the use of cranial nerve stimulation (including vagal nerve stimulation, VNS) in the treatment of various medical disorders including epilepsy, treatment resistant depression, anxiety disorders, chronic pain, migraine headaches, cardiac failure and cardiac arrhythmias. Treatments utilizing VNS may rely largely on surgically implanted medical devices designed to directly stimulate the vagal nerve in a manner similar to implanted cardiac pacemakers. These treatments are costly and may have adverse events. There is a need for a non-invasive safer alternative treatment modality to impact on the parasympathetic nervous system for widespread treatment of these disorders.

While there are multiple components of the autonomic system, it can primarily be divided into the sympathetic nervous system (SNS) and the parasympathetic nervous system (PNS). The sympathetic nervous system enables flight and fright bodily responses for emergencies and stress. The parasympathetic nervous system allows us to rest and digest; the sympathetic nervous system can be considered a quick response, mobilizing system and the parasympathetic a more slowly activated dampening system.

One manner in which the autonomic nervous system can be modulated is through the primitive autonomic nervous system reflex known as the diving reflex. The diving reflex is triggered by immersion of the body in cold water, and is characterized by a reduction in heart rate (HR) due to an increase in cardiac vagal activity, a primary efferent of the parasympathetic nervous system; this is often associated with vasoconstriction of selected vascular beds, due to increased sympathetic output to the periphery. The diving response is considered the most powerful autonomic reflex known. Diving bradycardia has been widely investigated and discussed by physiologists. Medical devices that elicit this reflex for the treatment of medical disorders via activation of the parasympathetic nervous system are not known. A medical device that stimulates the parasympathetic nervous system utilizing this natural reflex could have therapeutic applications in medical disorders where enhancing the parasympathetic nervous system has been shown to be therapeutic.

Diving bradycardia occurs in all air-breathing vertebrates, from amphibians to mammals. The diving reflex represents a subgroup of trigemino-vagal reflexes, together with the trigemino-cardiac reflex and the oculo-cardiac reflex.

A complex neural network integrating the respiratory and cardiovascular systems controls the diving response. Initiation of this reflex results primarily from stimulation of receptors on trigeminal afferent fibers, particularly those located in the forehead, periorbital region and the nasal passages. Cold receptors appear to be mainly involved in initiation of the diving reflex. In this regard, the stimulation of cold receptors in the skin of parts of the body other than the face does not result in slowing of HR. That the central circuit of the diving reflex is intrinsic to the brainstem is demonstrated by the fact that the bradycardic response is also maintained in de-cerebrated preparations. The physiological background of this circuit has been the subject of very few investigations. Some data suggest that the first relay of the circuit may be located in the ventral superficial medullary dorsal horn, as the cardiac responses can be blocked by the injection of either lidocaine or kinurenic acid. Thus, vagally mediated bradycardia and sympathetically mediated vasoconstriction may be mediated by the trigeminal system within the lower brainstem. However, the connections between the trigeminal system and autonomic neurons of the brainstem are unknown.

The human diving response involves bradycardia, often leading to a decrease in cardiac output (CO) and vasoconstriction of selected vascular beds, increasing blood pressure (BP) and reducing blood flow to peripheral capillary beds. The diving response in humans can be simulated by immersion of the face in cold water; this laboratory procedure is known as ‘simulated diving response’ or ‘cold pressor test’ and most knowledge of the diving response has been obtained by means of this procedure. The direct contact of cold water with the forehead, eyes and nose is sufficient to elicit the bradycardic response. The bradycardic response to apneic face immersion is highly variable among individuals; the reduction in HR generally ranges from 15 to 40%, but a small proportion of healthy individuals develop bradycardia below 20 beats/min. The reduction in HR is prevented by pretreatment with atropine, which demonstrates the role played by the vagal system. The increase in BP is also highly variable among healthy individuals. Similar variable reductions in HR have been observed after whole-body immersion; HR declines just after immersion, and then tends to remain stable, but it may decline to 20-30 beats/min during prolonged dives. If the ‘struggle phase’ is reached, HR further decreases and systolic BP can rise to 220-300 mmHg. After re-emersion, HR and BP normalize fairly rapidly.

Most evidence shows that the temperature of both water and air has significant effects in opposite directions on the magnitude of diving bradycardia: the lower the water temperature and the higher the air temperature, the more pronounced the bradycardic response. Facial cold receptors are most strongly excited by immersion in cold water. However, whole-body immersion in very cold water (˜0 C) can induce a paradoxical response; that is, tachycardia instead of bradycardia, the so-called ‘cold shock response’. This very probably involves a large afferent drive from cutaneous cold receptors, which stimulates the sympathetic system.

The vagal system has been shown to be the primary efferent neural pathway for cardiac adjustment in animals. After pretreatment with atropine, HR was high and did not change during dives. Moreover, in seals, marked oscillations of HR (10-20%) have been observed after immersion, which are an expression of a high vagal tone.

Several lines of research show that VNS has therapeutic activity in various medical disorders. Chronic, intermittent VNS as an adjunct to anti-epileptic drug therapy (AED) has been well documented as a treatment option for patients with refractory seizures that provides a significant reduction in seizure frequency and severity, as well as an improvement in QOL.

VNS has been shown to be beneficial in the treatment of treatment resistant depression. In one study, a 42% response rate after 2 years therapy was observed. A similar study showed a 53% response rate after 1 year therapy. Possible mechanisms of action include an overall increase in firing of brainstem cell body nuclei for 5-HT and NE neurons.

It would be beneficial to provide devices and methods for treating a patient that may non-invasively apply region cooling to specific regions in order to modulate the parasympathetic nervous system, including evoking a diving reflex-like response. Described herein are methods and apparatuses that may address these needs.

### SUMMARY OF THE DISCLOSURE

Described herein are novel medical device designed to specifically cool the head (and in particular, a region of the forehead), to a specific temperature range to achieve a therapeutic effect. Any of the methods and apparatuses described herein may alternatively or additionally include thermal application to the forehead area, nose, nasal passages and eye area as separate application sites as well the various combinations of these sites. Additionally or alternatively, the apparatus and method may be configured to cover (and thermally regulate) the entire face and/or the entire head area to the same or different temperatures. Alternatively or additionally, other body locations having cold sensitive nerve receptors that can stimulate a parasympathetic response may be used, for example the back of the neck (where nerve may be close to the surface of the skin, providing good access).

Forehead cooling may provide an indirect path towards activating the parasympathetic nervous system. A medical device, such as those described herein, that produce regional cooling on the face may impact on medical disorders that have shown improvement with VNS.

For example, described herein are methods of non-invasively increasing activity of the parasympathetic nervous system in a patient. Any of these methods may include: applying cooling from a thermal applicator to a region of the patient's face that is innervated by the trigeminal nerve; monitoring heart rate variability (HRV) in the patient while applying the cooling; and adjusting the cooling based on the HRV so that the HRV remains elevated relative to a patient baseline while cooling; and maintaining the cooling for at least 15 minutes.

Cooling may be applied, for example, to a region of the forehead along the midline of the forehead. For example, cooling may be applied to a region of the midline of the forehead having a diameter of less than 6 cm. Applying cooling may comprise applying to a region innervated by the maxillary nerve (V2) of the trigeminal nerve (e.g., the side of the face between the eye and the ear on one side of the face). For example, applying cooling may comprise applying to a region innervated by the ophthalmic nerve (V1) of the trigeminal nerve (e.g., the forehead region above the eyebrows extending upwardly towards the hairline and/or over the crown of the front of the head). Alternatively or additionally, applying cooling may comprise applying to a region innervated by the mandibular never (V3) of the trigeminal nerve (e.g., the side of the jaw). Applying cooling may comprise applying from an applicator adhesively attached to the patient's face.

Monitoring heart rate variability (HRV) may include monitoring high-frequency HRV. Alternatively or additionally, monitoring HRV may include monitoring from a sensor on the thermal applicator. Any of the methods and apparatuses described herein may include gathering a baseline HRV from the patient before applying cooling. The baseline may be measured from the patient before applying cooling (e.g., for a predefined amount of time, e.g., 30 seconds, 1 minute, 2 minutes, 3 minute, 4 minutes, 5 minutes, 6 minutes, 7 minutes, 8 minutes, 9 minutes, 10 minutes, 15 minutes, etc.). The baseline may be an average. In some variations the baseline may be collected and stored in a memory of the device for later use/comparison.

As mentioned, the cooling may be adjusted based on the HRV. For example, the cooling may be adjusted as a closed feedback loop. For example, the temperature may be maintained within a predefined range (e.g., between 5 degrees C. and 22 degrees C., between 5 degrees C. and 20 degrees C., between 5 degrees C. and 18 degrees C., between 10 degrees C. and 20 degrees C., between 10 degrees C. and 18 degrees C., between 10 degrees C. and 15 degrees C., between 5 degrees C. and 15 degrees C., etc.) and the temperature raised or lowered within this range based on the HRV (e.g., if the HRV is not elevated above the baseline HRV by a predefined amount (e.g., 5%, 10%, 15%, 20%, 25%, 30%, 35%, 40%, 45%, 50%, 60%, 70%, 80%, 90%, etc.) the temperature of the temperature of the thermal applicator may be lowered within this range. For example, the temperature of the thermal applicator may be lowered if the HRV is not elevated above a predetermined threshold compared to the patient baseline while cooling. In some variations, adjusting the cooling based on the HRV comprises adjusting the cooling between about 10-15 degrees C.

In general, the temperature of the thermal applicator may be held (maintained) at a predefined temperature (or temperature range, e.g., between about 5° C. about 15° C., between 10° C. and about 15° C., etc.) for some predetermined amount of time (e.g., 5 minutes or more, 7 minutes or more, 8 minutes or more, 9 minutes or more, 10 minutes or more, 12 minutes or more, 15 minutes or more, 20 minutes or more, 25 minutes or more, 30 minutes or more, etc.). For example, cooling may be maintained for at least 15 minutes, for at least 30 minutes, etc.

Any of these methods may include repeating the steps of applying, monitoring, adjusting and maintaining at least twice a day for 10 days (e.g., 2× more per day for at least 10 days, 12 days, 14 days, 15 days, 20 days, 21 days, 25 days, 28 days, etc.).

Any of these methods and apparatuses described herein may be configured to treat a neurological disorder. For example, maintaining the cooling (e.g., for at least 15 minutes) may include treating the patient for a neurological disorder by maintaining the cooling for at least 15 minutes, wherein the neurological disorder is one or more of: depression, attention deficit hyperactivity disorder (ADHD), epilepsy, and migraines.

For example, described herein are methods of non-invasively increasing activity of the parasympathetic nervous system in a patient, the method comprising: collect a baseline heart rate variability (HRV) for the patient; applying cooling from a thermal applicator to a region of the patient's face that is innervated by one or more of: the maxillary nerve of the trigeminal nerve; the ophthalmic nerve of the trigeminal nerve; and the mandibular nerve of the trigeminal nerve; monitoring HRV in the patient while applying the cooling through one or more sensors on the thermal applicator; and adjusting the cooling based on the HRV so that the HRV remains elevated relative to a patient baseline while cooling; and maintaining the cooling for at least 15 minutes.

In any of the methods and apparatuses described herein, the method of applying cooling may be limited to a region of the head/face that is small (e.g., less than about x cm², such as less than 5 cm², less than 6 cm², less than 7 cm², less than 8 cm², less than 9 cm², less than 10 cm², less than 12 cm², less than 15 cm², less than 20 cm², less than 25 cm², less than 30 cm², etc.) For example, a method of selectively modulating a patient's trigeminal nerve by regionally cooling the trigeminal nerve may include: applying cooling to a 6 cm (or less) wide region around the midline region of the patient's forehead that is innervated by the ophthalmic nerve of the trigeminal nerve; maintaining cooling for greater than 15 minutes at between 10-15° C. to non-invasively increasing activity of the parasympathetic nervous system in the patient.

Applying may comprise applying from an applicator adhesively attached to the patient's face. Monitoring may include monitoring heart rate variability (HRV); for example, monitoring may include monitoring high-frequency heart rate variability (HRV). Monitoring heart rate variability (HRV) may comprise monitoring HRV from a sensor on a thermal applicator attached to the patient's forehead. As mentioned, any of these methods and apparatuses may include gathering a baseline heart rate variability (HRV) from the patient before applying cooling.

As already mentioned, cooling may include adjusting the cooling using an open-loop feedback, based on the heart rate variability (HRV) by lowering the temperature of a thermal applicator on the patient's forehead if the HRV is not elevated above a predetermined threshold compared to the patient baseline while cooling. Adjusting the cooling based on the HRV may include adjusting the cooling between about 10-15 degrees C. As also mentioned above, maintaining the cooling for at least 15 minutes may comprise maintaining the cooling for at least 30 minutes.

Any of these methods may include repeating the steps of applying and maintaining at least twice a day for 10 days. Thus, the dosing may be adjusted by increasing or decreasing the duration of time that cooling is applied, adjusting the temperature of the applied cooling, adjusting the number of times per day that the temperature is applied, etc.

As already mentioned, any of these methods may be used to treat a neurological disorder (e.g., depression, attention deficit hyperactivity disorder (ADHD), epilepsy, and migraines); for example, any of these methods may include maintaining the cooling for at least 15 minutes comprises treating the patient for a neurological disorder by maintaining the cooling for at least 15 minutes, wherein the neurological disorder is one or more of: depression, attention deficit hyperactivity disorder (ADHD), epilepsy, and migraines.

Also described herein are apparatuses that are adapted to perform any of the methods described herein. For example, described herein are apparatuses for selectively modulating the trigeminal nerve by cooling. The apparatus may include: an applicator having a skin-contacting thermal surface, wherein the skin-contacting thermal surface is 6 cm or less in diameter (e.g., maximum diameter of the skin-contacting surface and/or thermal surface contacting the skin); one or more sensors adjacent to or within the skin-contacting thermal surface, wherein the one or more sensors are configured to sense heart rate; an adhesive adjacent to the skin-contacting thermal surface of the thermal applicator to secure the skin-contacting thermal surface to a patient's skin; and a thermal control configured to control the temperature of the skin-contacting thermal surface at between about 10-15 degrees C.

In some variations, the apparatus may include a connector assembly configured to connect a thermal applicator (surface) to a cooling unit, wherein the thermal control is contained at least partially within the cooling unit. The apparatus may also include a user interface on the cooling unit, wherein the user interface is configured to allow the user to control the application of cooling. The apparatus may include a fluid cartridge configured to deliver cooled, temperature-controlled fluid to the cooling surface of the applicator.

Described herein are apparatus for non-invasively increasing activity of the parasympathetic nervous system in a patient, which may be used to treat a disorder such as one or more of depression, ADHD, epilepsy, anxiety, chronic pain, migraines, heart failure and/or cardiac arrhythmia. The apparatus may include: an applicator having a skin-contacting thermal surface that is configured to attach to a region of the patient's face that is innervated by the patient's trigeminal nerve to apply cooling; one or more heart rate (HR) sensors on the applicator, the one or more HR sensors configured to measure the patient's heart rate; control circuitry configured to control the temperature of the skin-contacting thermal surface between 10-15 degrees C. and further configured to monitor heart rate variability (HRV) in the patient while applying the cooling from the applicator, wherein the control circuitry is further configured to adjust the temperature of the skin-contacting thermal surface based on the patient's HRV.

The control circuitry may include a processor, memory, power supply (which may be rechargeable, e.g., battery, inductive power supply, etc.) and may be configured to adjust the temperature of the skin-contacting thermal surface based on the patient's HRV so that the HRV remains elevated relative to a patient baseline while cooling the skin-contacting thermal surface.

The skin-contacting thermal surface may be 6 cm or less in diameter (e.g., at largest diameter).

The one or more heart rate sensors are adjacent to or within the skin-contacting thermal surface. Any appropriate sensor may be used, including electrical sensors (e.g., electrodes, photodiodes/emitters, piezoelectric sensors, etc.). The sensor may be connected to the control circuitry to extract HR and/or HRV (including high-frequency HRV) information from it, or it may be self-contained and able to detect and transmit HR and/or HRV to the control circuitry. A self-contained sensor may include a detection circuit, filtering circuit, digitizing circuitry (e.g., analog-to-digital converter), or the like; alternatively this may be included in the control circuitry.

The apparatus may include an adhesive adjacent to the skin-contacting thermal surface of the thermal applicator to secure the skin-contacting thermal surface to a patient's skin. The skin-contacting thermal surface may be shaped to optimally contact the region over a portion of the trigeminal nerve, such as the ophthalmic nerve of the forehead. For example, the skin-contacting thermal surface may be tapered at a bottom and wider at the top.

The apparatus may include a connector assembly connecting the applicator to a cooling unit housing the control circuitry. The connector assembly may include a wired connection (e.g., transmitting sensor data) and/or tubes for fluid transfer (e.g., passing chilled fluid between the cooling unit and the applicator to chill the skin-contacting thermal surface. For example, in some variations, the apparatus includes a fluid configured to circulate through a channel in thermal communication with the skin-contacting thermal surface, wherein the temperature of the fluid is controlled by the control circuitry.

The cooling unit may include a user interface, wherein the user interface is configured to allow the user to control the application of cooling (e.g., start treatment, stop treatment, display treatment parameters such as sensor output, temperature applied, patient compliance, etc.).

Alternatively or additionally, in some variations, the apparatus is configured to be wireless, so that the control circuitry, which may be integrated into the applicator or may be part of a separate cooling unit, wirelessly communicate with a remote processor (e.g., phone, pad, tablet, computer, server, etc.).

In general, any of these apparatuses may include a thermoelectric cooler (TEC) in thermal communication with the skin-contacting thermal surface in thermal communication with the skin-contacting thermal surface either directly or indirectly. In some variations the TEC is part of a separate cooling unit that is connected to the applicator by a connector assembly; a thermal transfer fluid may be cooled and used to cool the skin-contacting thermal surface. In some variations, a TEC is part of the applicator; for example, the TEC may be positioned adjacent to the skin-contacting thermal surface on the applicator.

The skin-contacting thermal surface of the applicator may be adapted to cool one or more regions of the trigeminal nerve. For example, the skin-contacting thermal surface of the applicator may have a substantially crescent shape and the applicator may be configured to adhesively attach to the side of the patient's face between the patient's eye and the patient's ear to cool the patient's maxillary nerve. In some variations, the skin-contacting thermal surface of the applicator has an angled shape, having an angle of between 100 and 135 degrees, and the applicator is configured to adhesively attach to the side of the patient's jaw to cool the patient's mandibular nerve.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows an examples of one variations of a system for cooling including an applicator and a cooling unit. In this example, the applicator may be worn over the patient's forehead.

FIG. 2 illustrate the applicator of FIG. 1 worn on the patient's forehead while lying down.

FIG. 3A is a front view of a cooling unit.

FIG. 3B is a back view of the cooling unit.

FIG. 3C is top perspective view of the cooling unit.

FIG. 4 is an overview of a system as described herein.

FIG. 5 is a drawing and photograph of headgear and forehead pad.

FIG. 6 is a close up view of an example of a forehead pad.

FIGS. 7-9 illustrates the effect of localized, non-invasive cooling of a subject's trigeminal never (applied to a region of the patient's forehead innervated by the ophthalmic nerve branch of the trigeminal) showing the change in average heart rate (FIG. 7), systolic blood pressure (SBP, FIG. 8) and diastolic blood pressure (DBP, FIG. 9) during baseline (sitting and reclining), onset of cooling and cooling (reclining) and recovery (reclining) conditions. Data are averaged in 30 s epochs.

FIG. 10 is a graph showing the average percentage change of several autonomic indices across consecutive 5 min periods during the start and maintenance of the application of cooling (calculated as change from the reclining baseline, in 5 min blocks) to a region of the patient's forehead innervated by the ophthalmic nerve branch of the trigeminal.

FIGS. 11A and 11B show heart rate variability and high frequency heart rate variability indices, respectively, calculated in subsequent 5 min periods across baseline (sitting and reclining), during the application of cooling (“EBB”) while reclining and during recovery (reclining) conditions.

FIG. 12 is a graph showing the heart rate profiles for the two subjects who remained awake during the application of cooling from the samples analyzed in FIGS. 10-11B.

FIG. 13 is a graph showing a time course of the cooling treatment from a cooling applicator (such as the one shown in FIG. 14A-14B) look at the temperature of the skin-contacting thermal surface of the applicator over time during the application of therapy. The temperature gradually decreasing over time.

FIGS. 14A and 14B show front and side views, respectively, of a patient wearing an example of an applicator having a skin-contacting thermal surface that is 6 cm or less in diameter, and includes one or more sensors adjacent to or within the skin-contacting thermal surface (e.g., sensors configured to sense heart rate, HRV, etc.). The applicator is part of a cooling apparatus and is adhesively attached to the patient's skin at a midline portion of the forehead, covering a region of the skin that is innervated by the trigeminal nerve (sensor nerves of the ophthalmic nerve of the trigeminal).

FIG. 15 is a schematic of an apparatus including an applicator such as shown attached to the subject in FIGS. 14A-14B.

FIGS. 16A-16B show front and side views, respectively, of a patient wearing an example of an applicator having a skin-contacting thermal surface that is 6 cm or less in diameter, and includes one or more sensors adjacent to or within the skin-contacting thermal surface. In this example, the applicator is self-contained, and does not include a connection to separate cooling unit. For example the applicator may include a TEC.

FIGS. 16C-16D are bottom and side schematic views, respectively, of an applicator such as the one shown in FIGS. 16A-16B. In this example, the applicator includes an integrated cooling unit (e.g., thermoelectric cooler, TEC, such as a politer cooler), rechargeable power supply, control circuitry, heat transfer vents and adhesive.

FIGS. 17A-17B illustrate another example of an applicator (which may be connected to a cooling unit as in FIG. 15, or may be self-contained, as in FIGS. 16A-16D) shown in a front and side views, respectively, when worn by the patient. In this example, the applicator is configured to be worn on the side of the face, e.g., between the ear and the eye, to apply cooling to the maxillary nerve of the trigeminal providing sensory innervation of the face in this region.

FIGS. 18A-18B illustrate another example of an applicator (which may be connected to a cooling unit as in FIG. 15, or may be self-contained, as in FIGS. 16A-16D) shown in a front and side views, respectively, when worn by the patient. In this example, the applicator is configured to be worn on the side of the face, e.g., along the side of the jaw, to apply cooling to the mandibular nerve of the trigeminal providing sensory innervation of the face in this region.

FIGS. 19A-19D illustrate example of applicators that may be used (and may be self-contained or may connect to a separate cooling unit). In this example, the applicator is adapted for use on a region of the forehead. FIG. 19A is an example of an applicator that is broader at the top region than at the bottom region, which may allow application of cooling to the trigeminal nerve underlying the skin without substantially cooling the facial nerve, for example, when the applicator is worn on the midline of the forehead. FIG. 19B shows another example, in which bottom end region is tapered and smaller than the upper end. FIG. 19C is an example of an oval applicator, while FIG. 19C is another example of a tapered-bottom applicator, similar to FIG. 19A.

FIG. 20 is an example of a method of non-invasively increasing activity of the parasympathetic nervous system in a patient.

FIG. 21 is a schematic of a method of selectively modulating the trigeminal nerve by cooling.

### DETAILED DESCRIPTION

In general, described herein are apparatuses for non-invasively applying cooling to modulate the parasympathetic nervous system by cooling the patient's face in regions overlying the trigeminal nerve (e.g., the ophthalmic nerve, the maxillary nerve and/or the mandibular nerve). Applying cooling to the nerve may trigger a diving reflex-like response, increasing activity of the parasympathetic nervous system. In some variations, the cooling may be regulated by a feedback mechanism, which may include monitoring one or more indicators of parasympathetic activity, including heart rate (HR) and/or heart rate variability (HRV), and in particular high frequency HRV. Cooling may be applied in a limited manner, to avoid cooling regions innervated by, e.g., the facial nerves.

In some variations, the cooling may be applied by a small applicator that is part of an apparatus (e.g., system, device, etc.) that is adapted specifically to modulate the parasympathetic nervous system (e.g., increase parasympathetic activity) and/or treat an indication such as (but not limited to) depression, attention deficit hyperactivity disorder (ADHD), epilepsy, and migraines.

Although any device that may apply local cooling of the region innervated by the trigeminal nerve may be used, in some variations it may be particularly beneficial to apply cooling to regions that have a skin-contacting surface area that is limited to less than, e.g., 60 cm² (e.g., less than 50 cm², less than 45 cm², less than 40 cm², less than 35 cm², less than 30 cm², less than 25 cm², less than 20 cm², less than 15, less than 10 cm², less than 9 cm², less than 8 cm², less than 7 cm², less than 6 cm², less than 5 cm², etc.). Highly regional cooling may, surprisingly, still evoke a diving-like reflex, may require less power, and may be more easily tolerated by the subject (e.g., patient).

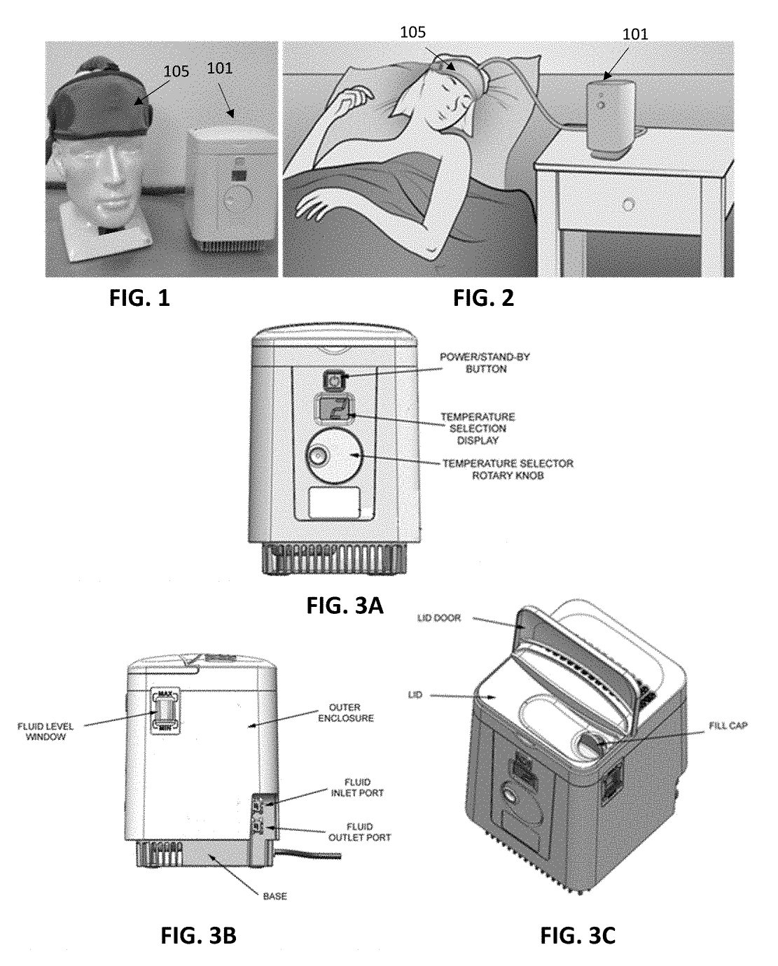

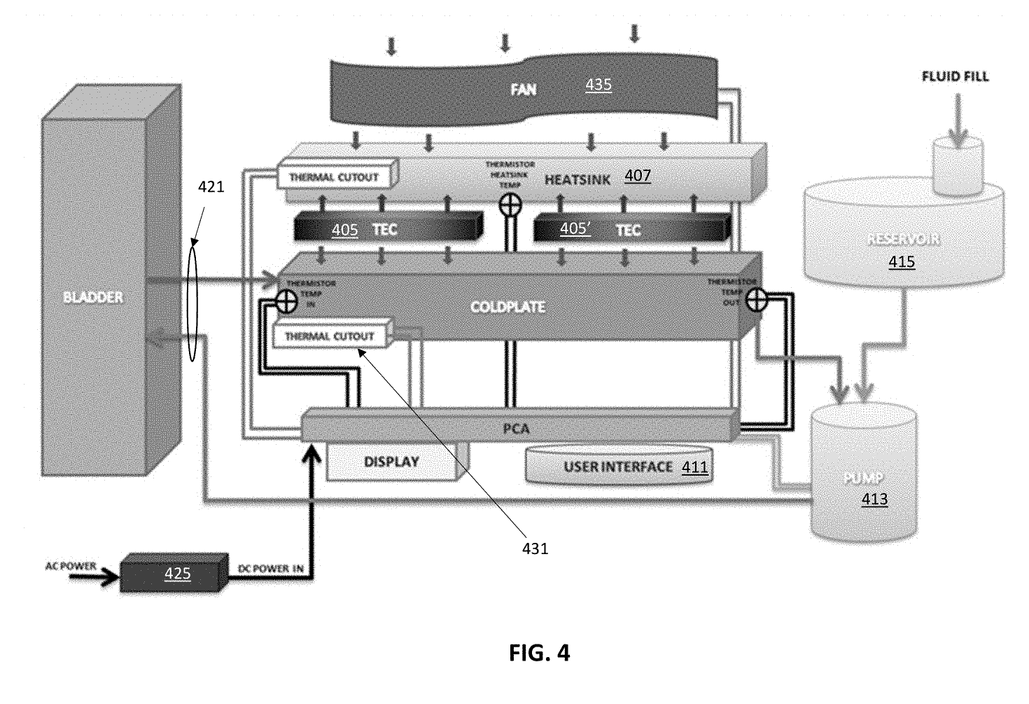

FIGS. 1 and 2 illustrate an example of a system that may apply cooling to a patient's forehead. In FIG. 1, the system includes three components: a cooling unit (e.g., configured in this example as a bedside unit) 101, the forehead pad (not visible), and headgear 105. The cooling unit in FIGS. 1-4 is shown as a separate device (e.g., “bedside” unit) that may cool a fluid and transport the fluid from the unit to the applicator pad (e.g. the skin-contacting thermal surface of the applicator). The cooling unit in this example utilizes a solid-state thermoelectric device 405 (or multiple devices 405′) to cool a thermal transfer fluid 415 consisting of purified water and alcohol. This is illustrated in the schematic of FIG. 4. The unit may include a user interface 411 that allows the user to turn the unit on and off, and adjust the temperature (e.g., +/−2° C. The unit contains a pump 413 for circulating the thermal transfer fluid through the tubing 421 and forehead pad. The bedside unit is powered by a DC electrical power supply 425 and is controlled by an integral control unit (CU) (control circuitry 431, also referred to as a controller) and its firmware. The CU controls the cooler, pump and fan 435 by providing pulse-width modulation (PWM) of the DC power to each component according to feedback inputs sensed by thermistors.

FIGS. 3A-3C illustrate one embodiment of a prototype cooling unit similar to that shown in FIGS. 1-2. In this example, the applicator may be connected to the cooling unit by a connector (e.g., tubing) that allows fluid to pass from the cooling unit and circulate within the applicator to cool the skin-contacting thermal surface.

The system may regulate the temperature of fluid to a temperature set point within a few minutes (e.g., 5 minutes, 7 minutes, 10 minutes, 12 minutes, 15 minutes, 20 minutes, etc.) of setting. The fluid temperature may be set between, e.g., 8 and 18° C. (which may allow the applicator temperature to be controlled to approximately this temperature; the system may account for heat loss in transmitting the fluid to the applicator. The apparatus may include a cartridge with the fluid, and may include built-in safety mechanisms that mitigate the risk of any type of fault condition of the cooling unit or any of its components.

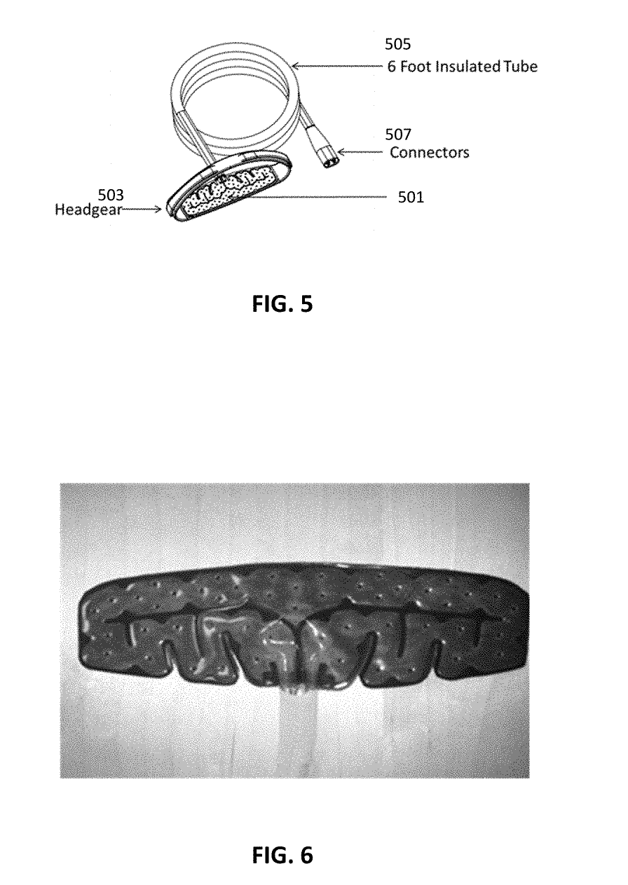

The applicator may contain the wearable portion of the apparatus and may be strapped and/or adhesively attached to the patient's face. In some variations (such as those shown in FIGS. 1-2 and FIG. 5) it may include a forehead pad 501 that is in contact with the patient's head, the headgear 503 that holds the forehead pad in place and a length of insulated tubing 505 (e.g., six feet) that connects via a connector 507 to a cooling unit. The applicator generally includes a thermal transfer pad (e.g., skin-contacting thermal transfer pad) that may be configured, including being shaped, to cover the target area of the face. In variations in which the skin-contacting thermal transfer surface (which may be an outer surface of the skin-contacting thermal transfer pad) is configured to be cooled by a circulating fluid the pad may include a tortious channel (or channels) through which the fluid moves. FIG. 6 is an example of one variation of a pad that is configured to be worn on the forehead and includes a fluid path within it. In this example, the pad is fabricated from a urethane film sheet, Bayer PT9200. In some variations the pad may be held in a holder, e.g., a headgear. The holder or headgear may provide a mechanism to hold the pad in position on the user's face for cooling. For example the headgear may be fabricated from a fabric material, such as a Lycra based material.

In some variations the apparatus the skin-contacting thermal transfer surface may also include a hydrogel allowing for increased surface area contact and increased thermal transfer characteristics.

In one arrangement of the device, the thermal transfer pad is shaped to cover the region of the forehead that overlies glabrous (non-hairy) skin in a region that is innervated by the sensor nerves of the trigeminal cranial nerve. This region may be important for providing temperature information to elicit a diving reflex like response (which may in turn result in a vagal response) given that it has the highest thermal sensitivity of body surfaces and it has a neural and vascular supply that are specialized for this function. The forehead has unique physiological and neuroanatomical properties and may play a prominent role in influencing the diving reflex. The distribution of warm and cold spots has been shown to be highest over the face and forehead of all body parts. Thermal sensation has been shown to be highest in the forehead. Thermal sensitivity of the face, as measured by its effect on sweating rate change from the thigh, is approximately three times that of the chest, abdomen men and thigh. As described herein, the application of a cooling stimulus at the scalp on the forehead may be associated with alterations in the parasympathetic nervous system that may be therapeutic in a variety of medical disorders via reflex activation of the parasympathetic nervous system. Altering skin temperature on one or more regions of the forehead, therefore, may be a very sensitive and non-invasive manner to treat medical disorders known to be impacted by vagal nerve stimulation.

The methods and apparatuses described herein may generally be used to treat disorders in which there is parasympathetic component. By applying localized cooling and evoking a diving-like reflex, the parasympathetic system may be modulated, which may beneficially be used to treat a variety of indications. In particular, neuropsychologial indications that may be treated by the method and apparatuses described herein include depression and attention deficit hyperactivity disorder (ADHD), anxiety disorders, and pain. For example, these method may be used to treat depression, and particularly treatment-resistant depression to improve mood in depressed patients. The methods and apparatuses may be used to improve anxiety in anxiety disorder patients. These methods and apparatuses, or a modified version of them, may also be used to improve mood and anxiety symptoms in other neuropsychiatric disorders such as, but not limited to, substance abuse, post-traumatic stress disorder, psychotic disorders, manic-depressive illness and personality disorders and any neuropsychiatric patient who experience mood or anxiety problems. Pain may be treated (including chronic pain, and headaches, including migraine headaches. Mood and anxiety may be treated in patients with mood or anxiety problems secondary to other medical disorders such as cardiac, endocrinological, and pulmonary disorders.

The use of the local cold therapy applied to the face (and in particular, applied noninvasively to sensory neurons of one or more branches of the trigeminal nerve, such as the ophthlamlic nerve, maxillary nerve, and/or mandibular nerve) may be used to treat a disorder such as a neuropsychiatric disorder in a subject by evoking the diving reflex-like response in a controlled manner. Cooling a region of the face innervated by a portion of the trigeminal nerve (e.g., the ophthalmic nerve, the maxillary never and/or the mandibular nerve) to 15 degrees C. or less (e.g., between 10-15° C.) following onset of a neuropsychiatric episode may substantially reduce the duration and/or intensity of the episode. In addition, regular (e.g., daily, 5× weekly, 4× weekly, 3× weekly, 2× weekly, 1× weekly) treatment, e.g., for greater than 10 minutes (e.g., between 10-90 minutes, between 10-60 minutes, between 10-45 minutes, between 10-40 minutes, between 10-35 minutes, between 10-30 minutes, between 10-25 minutes, between 10-20 minutes, etc.) per session may reduce or prevent the occurrence and/or severity of an episode. Treatment may be more than 1× per day (e.g., 2× per day, 3× per day, 4× per day, etc.).

The methods and apparatuses described herein may also or alternatively be used to treat one or more of: epilepsy, treatment resistant depression, anxiety disorders, chronic pain, migraine headaches, heart (e.g., cardiac) failure and cardiac arrhythmias.

For example, induction of the parasympathetic nervous system via the parasympathetic diving reflex activation from cold facial stimulation may be beneficial in the management of headache patients through downstream modulation of neural systems implicated in the development and treatment of headaches. Although not desiring to be bound by theory, the inventors have found that the use of the local cold therapy applied to the face (and in particular, applied noninvasively to sensory neurons of one or more branches of the trigeminal nerve, such as the ophthlamlic nerve branch) may be used to treat migraines in an awake subject. For example, cooling to 15 degrees C. or less (e.g., between 10-15° C.) following onset of a migraine may substantially reduce the duration and/or intensity of the migraine. In addition, regular (e.g., daily, 5× weekly, 4× weekly, 3× weekly, 2× weekly, 1× weekly) treatment, e.g., for greater than 10 minutes (e.g., between 10-90 minutes, between 10-60 minutes, between 10-45 minutes, between 10-40 minutes, between 10-35 minutes, between 10-30 minutes, between 10-25 minutes, between 10-20 minutes, etc.) per session may reduce or prevent the occurrence and/or severity of migraines in patients that suffer from chronic migraines. Treatment may be more than 1× per day (e.g., 2× per day, 3× per day, 4× per day, etc.).

Any of the methods and apparatuses described herein may also be useful for the treatment of sleep, including sleep-related disorders, such as insomnia.

The use of the device on the scalp in the region over the area of the forehead is expected to provide a parasympathetic signal via the diving reflex similar to the effects of vagal nerve stimulation. VNS has been shown to be useful in the treatment of the disorders mentioned above.

For example, the methods and apparatuses described herein may be useful to treat a cardiac disorder such as heart failure (HF) and arrhythmias. In some variations, the methods and apparatuses described herein may be used to noninvasively and thermally modulate the autonomic nervous system (e.g., the parasympathetic/sympathetic nervous system) in a subject (e.g., patient).

The autonomic nervous system plays a vital role in maintaining normal cardiac rhythm and rate. As one of the 2 branches of the autonomic nervous system, vagal nerves act as a restraining force to sympathetic excitation and are critical in balancing the cardiac autonomic control. Excitation of vagal nerves, either spontaneously or via electrical stimulation, produces profound systemic effects. Specifically for the heart, vagal excitation exerts negative chronotropic, dromotropic and inotropic effects. Since VNS has profound effects on the cardiac electrophysiologic substrate and arrhythmogenesis, it has the potential to provide therapeutic effects for certain cardiac arrhythmias. The application of cooling to trigger a diving reflex-like response (e.g., by selectively/locally applying cold therapy to the face, and in particular, applied noninvasively to sensory neurons of one or more branches of the trigeminal nerve, such as the ophthlamlic nerve branch) may be used to treat a patient for heart failure and/or arrhythmia.

VNS produces classic negative chronotropic effect on the pacemaker cells in the sinus node. This raises the possibility that a device that enhances vagal tone may be therapeutic in medical disorders involving sinus tachycardia.

VNS and Ach have negative dromotropic effects on the atrioventricular node (AVN) conduction. Since vagal activation exerts negative dromotropic effect on the AVN conduction, it has been utilized for termination of supraventricular tachycardia involving the AVN. The vagal excitation for this purpose has been achieved by physical manipulations known as vagal maneuvers. The Valsalva maneuver and carotid sinus massage are such means to activate the vagal nerves and evoke vagal negative dromotropic effect on the AVN conduction. These maneuvers have been utilized clinically to terminate paroxysmal supraventricular tachycardia, including atrioventricular nodal reentrant tachycardia (AVNRT) and atrioventricular reentrant tachycardia (AVRT). Stimulation of the vagal nerve through devices that induce the diving reflex has not previously been used clinically to terminate paroxysmal supraventricular tachycardia.

Sinus rate cannot be restored in a majority of atrial fibrillation (AF) patients, and ventricular rate control remains the only option in these patients. Using VNS to control ventricular rate during AF is a potential useful therapy. The AVN area, which is richly supplied with vagal nerves, has very selective innervation by vagal fibers projecting from a discrete epicardial fat pad (“the AV nodal” fat pad), located at the junction between the inferior vena cava and the left atrium, at the crux of the heart. Due to this unique arrangement of the cardiac vagal network, electrical selective AVN vagal stimulation (AVN-VS) is possible and has been effective in slowing ventricular rate during AF. VNS may be a novel therapy for modulation of the AV node transmission and may offer transient or longer term control of the ventricular rate in certain population of patients with AF.

Reduced vagal activity may be associated with increased risk for life-threatening arrhythmias, sudden death, and cardiac mortality. The ATRAMI (Autonomic Tone and Reflexes After Myocardial Infarction) study and the CIBIS II (Cardiac Insufficiency Bisoprolol Study II) demonstrated that diminished cardiac vagal activity and increased heart rate were powerful predictors of increased mortality in heart failure (HF). An increase in vagal tone may provide protective effect against ventricular arrhythmias. HF is associated with high incidence of ventricular arrhythmias, with about 80% or more of these patients have frequent ventricular premature beats, and about 50% have runs of non-sustained ventricular tachycardia. Furthermore, sudden death accounts for approximately half of all deaths in HF, and many of these sudden deaths are due to ventricular arrhythmias. Improving autonomic balance by sympathetic inhibition with clonidine has been shown to reduce ventricular arrhythmias in HF patients. Chronic VNS therapy may also be beneficial in HF. Ventricular arrhythmias may be reduce or by improving autonomic balance.

Heart Failure (HF) is a major cause of morbidity and mortality, despite advances in medical and device therapy. Autonomic imbalance, with excess sympathetic activation and decreased vagal tone, is an integral component of the pathophysiology of HF.

Without being by any particular theory, the use of the local cold therapy applied to the face (and in particular, applied noninvasively to sensory neurons of one or more branches of the trigeminal nerve, such as the ophthlamlic nerve branch) may be used to treat heart failure and/or arrhythmia in a subject by evoking the diving reflex in a controlled manner. For example, cooling a region of the face innervated by a portion of the trigeminal nerve (e.g., the ophthalmic nerve, the maxillary never and/or the mandibular nerve) to 15 degrees C. or less (e.g., between 10-15° C.) following onset of heart failure and/or arrhythmia may substantially reduce the duration and/or intensity of the cardiac event. In addition, regular (e.g., daily, 5× weekly, 4× weekly, 3× weekly, 2× weekly, 1× weekly) treatment, e.g., for greater than 10 minutes (e.g., between 10-90 minutes, between 10-60 minutes, between 10-45 minutes, between 10-40 minutes, between 10-35 minutes, between 10-30 minutes, between 10-25 minutes, between 10-20 minutes, etc.) per session may reduce or prevent the occurrence and/or severity of such cardiac events. Treatment may be more than 1× per day (e.g., 2× per day, 3× per day, 4× per day, etc.).

A diving reflex-like response may occur when at least a portion of the face is cooled to a range of, e.g., between 10° C. to 15° C. in most patients, although in some patients temperatures ranging from 0 degrees C. to 30 degrees C. may produce a parasympathetic response akin to the diving reflex. Because there is variation between individuals, it may be beneficial to monitor the patient and adjust the temperature so as to achieve a robust diving reflex-like response (e.g., altering the patient's parasympathetic nervous system). Thus, any of the methods and apparatuses described herein may be configured to monitor the patient in this manner. For example, a cold facial stimulus that produces a parasympathetic changes related to a vagal reflex may be detected by measuring a change in autonomic nervous system physiology and incorporate it into a feedback loop that then results in changes in the temperature. In this manner, the temperature applied can be adjusted in real time to achieve the desired physiological effect.

In one arrangement, a variable temperature with defined changes can be delivered across the period of use with the changes linked to feedback from changes in the physiology of the body across a period of use. Any of the following physiological measures may be monitored and temperature adjusted in real time according to the level of the physiological measure: alterations in the function of the autonomic nervous system such as heart rate (HR), blood pressure (BP), heart rate variability (HRV), galvanic skin response (GSR), skin temperature (either at the skin on the head underneath the device, or on skin at some other portion of the head not underneath the device, and/or peripheral skin temperature, and/or core body temperature, e.g., measured internally or by some external means), alterations in the EEG signal (which may be additionally useful for the management and monitoring of epilepsy), alterations in pain sensitivity, alterations in cardiac function related to arrhythmias and heart failure, alterations in neurochemical function related to mood and anxiety such as serotonin, norepinephrine and dopamine, etc.

In some variations, the apparatus and method may be configured to allow the patient wearing the device to modify the temperature across the period of use with the changes linked to subjective feedback. For example, a control on the device may allow the person wearing the device to adjust the temperature according to their immediate comfort and treatment needs, either up or down some small increments.

In the clinical management of a patient, a healthcare provider may want to know certain parameters of the patient and/or device over multiple nights of use such that care can be optimized. For example, any of the apparatuses and methods described herein may include patient compliance monitoring. Compliance monitoring may detect (e.g., based on the output of the one or more sensors, for example) when the apparatus is worn on the subject's body. Compliance data may be stored locally, analyzed, and/or transmitted.

In one arrangement that apparatus includes a memory card or memory chip, and may be configured to automatically record certain parameters and store them for later display by the healthcare provider.

In monitoring their own care, a patient may want to know certain parameters of the patient and/or device over multiple nights of use such that care can be optimized. In one arrangement of the device a memory may automatically record certain parameters and store them for later display and/or transmission. Thus, in some variations, this information could be transferred to a healthcare provider's office or some other central database via the phone or Internet or some wireless technology where someone could review the information and provide recommended adjustments in the treatment accordingly.

Examples of information that may be stored could include, but would not be limited to: temperature of the device (e.g., thermal applicator), skin temperature, core body temperature, measures of autonomic variability, alterations in the function of the autonomic nervous system as assessed by any method of autonomic nervous system assessment by someone skilled in the art (e.g., heart rate, blood pressure, heart rate variability, etc.), galvanic skin response, skin temperature, alterations in the EEG signal, alterations in pain sensitivity, alterations in cardiac function, alterations in neurochemical function related to mood and anxiety such as serotonin, norepinephrine and dopamine, etc.

### EXAMPLE

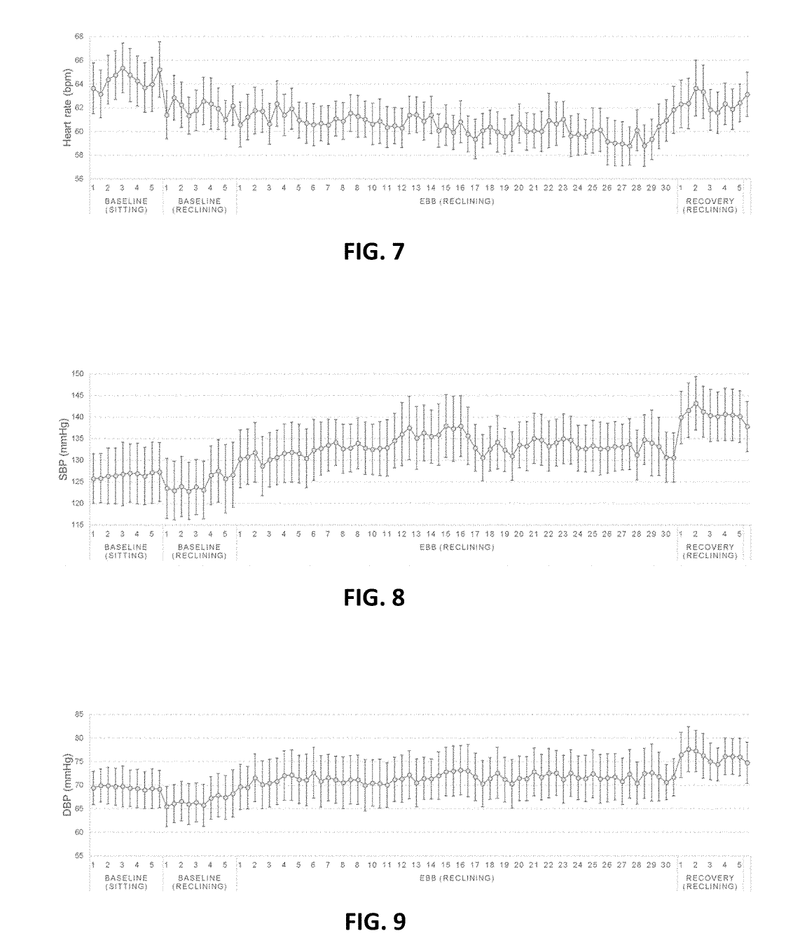

A study was made using the methods and apparatuses described above to assess the impact of non-invasive cooling to an external region of the face (e.g., a region of the forehead) in patients. The results of this preliminary study are shown in FIGS. 7-12. The apparatus used (as will be described in greater detail below) was found to impact the autonomic nervous system function in a reliable manner. The study focused measures of autonomic nervous system function acutely impacted by the use of the device in a 14-16° C. temperature setting.

The effect on the autonomic nervous system of patients was monitored over a continuous 2.5-hours period, including a pre-intervention baseline, wearing the device, and post-intervention recovery. Autonomic activity was continuously recorded. In addition, wearable devices were used to determine if sensitivity for assessing changes in autonomic measures when participants use the cooling device. Data was analyzed using standard lab protocols. Patient sample included a mix of gender, race and ethnicity and were healthy (e.g. no severe medical conditions such as cancer, heart disease, or diabetes; no sleep disorder other than insomnia; no DSMS psychiatric disorder, etc.). The in-lab study lasted about three hours in a private, comfortable, dimly-lit, sound-attenuated room with regulated temperature (68-72° F.). Ambient temperature was monitored during the recording. The participant were seated in a reclining chair throughout the recording. An initial baseline recording was made, after which the device was attached. Briefly, the pad was placed on the subjects' head at 30° C. (setting A). Five minutes after the application of the headgear and forehead pad to the subject, the temperature of the pad was set to 15° C. (Setting B at a level of “3”). There is a time delay between when the device is set in active mode and achieving the desired 15° C. (e.g., approximately 20-25 min in the experimental apparatus, see, e.g., FIG. 13). Participants wore the device for 30 min, after which it was removed. Recordings continued for a post-intervention (recovery) period.

The following measures were continuously recorded: Electrocardiogram (ECG): heart rate (HR, bpm) and standard measures of heart rate variability (HRV) in both frequency domain (e.g. total power (TP, ms2), low [LF, ms2] and high frequency [HF, ms2] power in conventional bands and arbitrary units) and time domain (e.g. standard deviation of normal to normal inter-beat intervals [SDNN, ms], Root Mean Square of the Successive Differences of normal to normal inter-beat intervals [RMSSD, ms]) approaches. From Impedance Cardiography (ICG): contractility indices (e.g. pre-ejection period [PEP, ms], total ejection period [TEP, ms], left ventricular ejection time [LVET, ms]). From Portapres: beat-to-beat systolic (SBP, mmHg) and diastolic (DBP, mmHg) blood pressure, pulse pressure (PP, mmHg). From Piezoelectric breathing bands: thoracic and abdominal respiratory rate (RR, breaths/min). From the combination of the above mentioned signals, additional indices may be calculated (e.g. baroreflex sensitivity). Standard sleep measures (electroencephalogram, EEG; electrooculogram, EOG; electromyogram, EMG), used to monitor the state of wakefulness in the individual were taken. EEG, ECG, EOG, EMG, and signals from the piezoelectric bands were recorded through Grael-PSG Units (Compumedics, Abbotsford, Victoria, Australia). Portapres Model-2 units (TNO TPD Biomedical Instrumentation, Amsterdam, NL) was used to noninvasively collect beat-by-beat blood pressure data with analog outputs interfaced with ExLink data Logger units (Compumedics, Abbotsford, Victoria, Australia). HIC-4000 Bioelectric Impedance Cardiographs (Bio-Impedance Technology, Inc., Chapel Hill, N.C.) was used for the non-invasive assessment of ICG contractility indices with analog outputs (Z0, dZ/dt) interfaced with the Grael-PSG Units. All signals were recorded through the ProFusion nexus platform and sleep scoring will be performed using ProFusion 3 (Compumedics, Abbotsford, Victoria, Australia). Participants were also be fitted with a wrist-worn wearable that measures activity and heart rate (e.g. Fitbit Charge) and a wearable placed on the finger that measures activity and photoplethysmography (PPG) (e.g. Oura ring) throughout the recording period. Also, subjective measures of relaxation, tension, fatigue, and sleepiness were assessed at 30 min time intervals across the recording period.

### Results

Across the baseline condition, as participants moved from a sitting to reclining position, there was a significant decrease in heart rate (HR) (p<0.05), together with a non-significant reduction in blood pressure (BP), possibly reflecting the change in posture (see, e.g., FIGS. 7-9). Pre-ejection period (PEP, an index reflecting cardiac sympathetic nervous system activity) also decreased across the baseline condition (p<0.05).

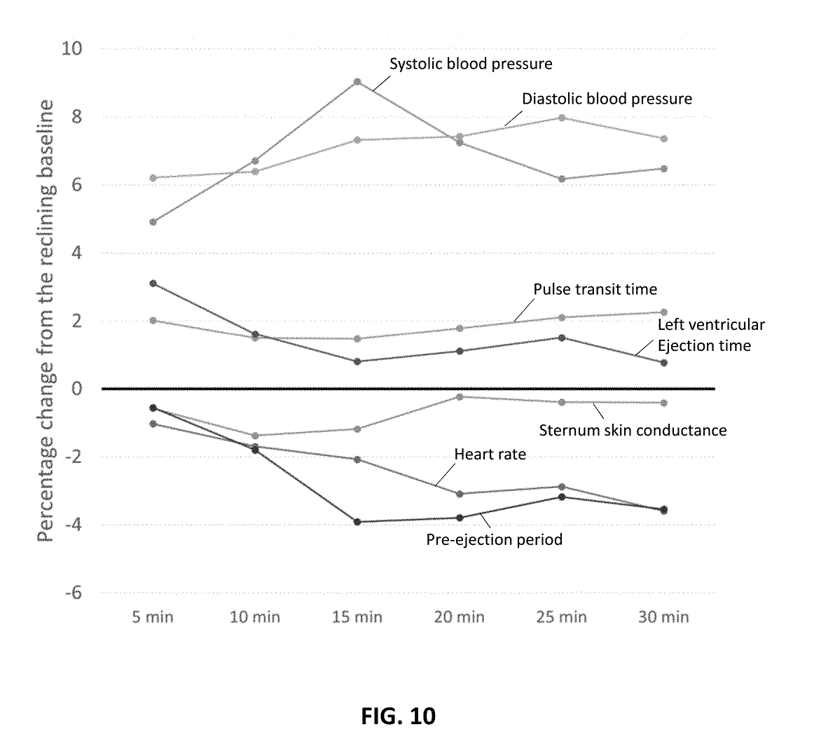

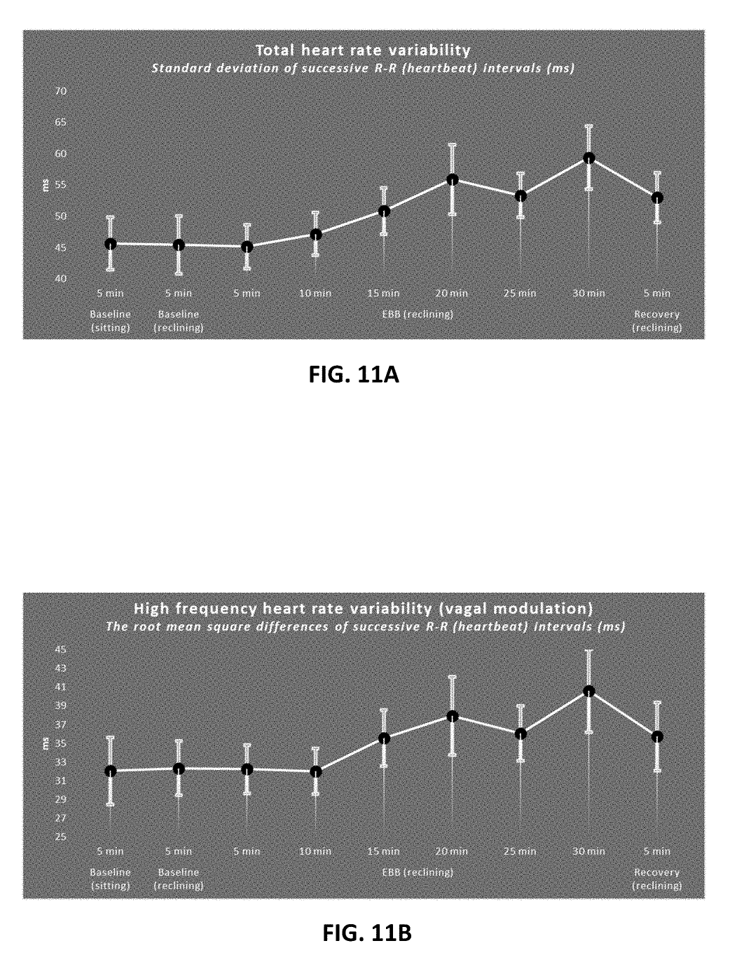

Across the cooling (e.g., “EBB”) condition, there was a progressive and significant drop in HR over time, matching a progressive increase in systolic and diastolic blood pressure (all p's<0.05) (see, e.g., FIGS. 8, 9 and 10). Total heart rate variability, measured by the standard deviation of successive R-R intervals (SDNN; reflecting the ability of the system to dynamically adjust to perturbation), and high frequency variability, calculated as the square root of the mean squared differences of successive R-R intervals (RMSSD; indicating vagal modulation), increased over time (all p's<0.05; FIGS. 11A-11B). No significant cooling-related changes were detected for the other indices (See, e.g., FIG. 10).

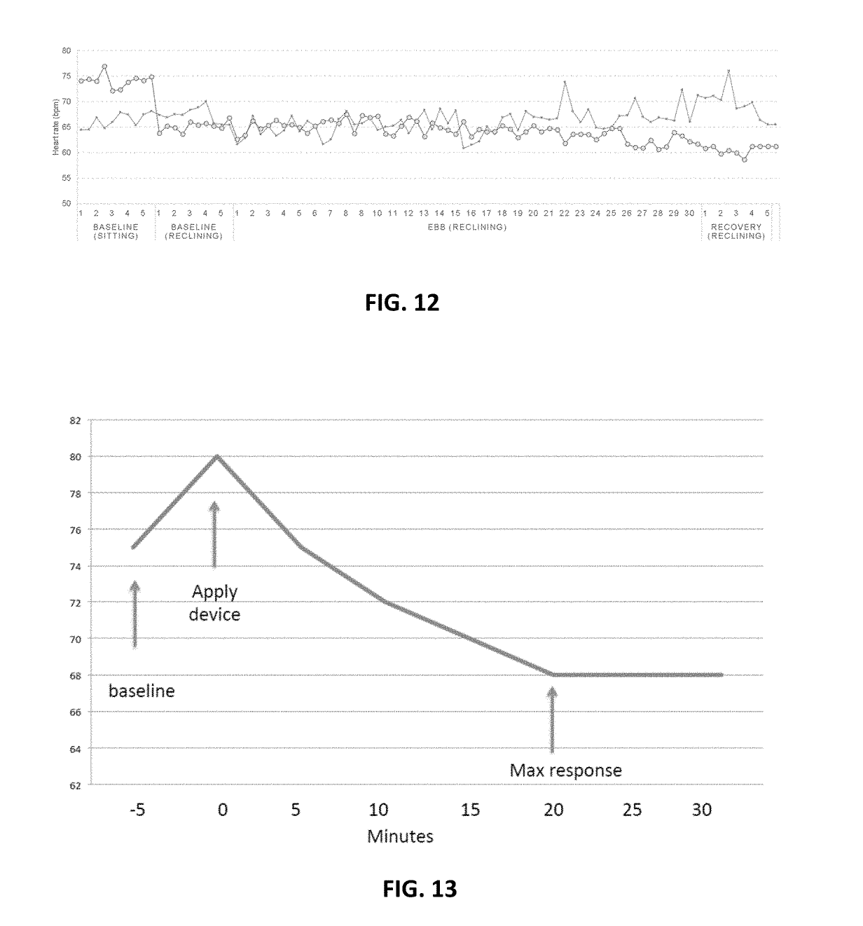

Some participants fell asleep at different time points within the experimental session. Participants who remained awake for the whole time also showed some evidence of a cooling-related drop in HR (FIG. 12).

At recovery, there was a sudden increase in HR, SBP, and DBP, possibly reflecting an awakening response (see, e.g., FIG. 9). To further explore this, we compared those participants who slept during the last 5 min of the EBB condition with those who did not. The magnitude of the physiological activation at recovery was bigger in those who were sleeping (see FIG. 10). PEP is an index reflecting sympathetic activity (shorter PEP reflects high sympathetic activity). There was suggestive evidence for a progressive decrease in PEP.

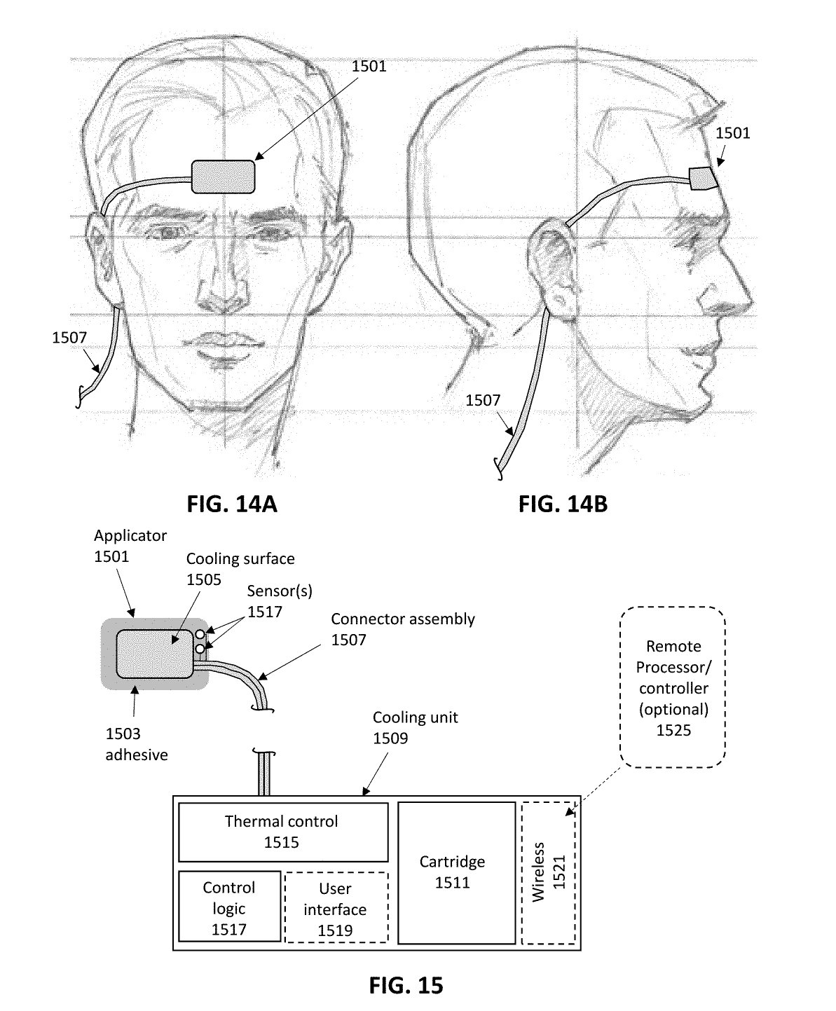

FIGS. 14A-14B illustrate one example of an apparatus for selectively modulating the trigeminal nerve by cooling. In FIG. 14A, the apparatus includes an applicator 1501 that includes a skin-contacting cooling surface that is placed against the forehead of the patient near a midline region. In this example, the apparatus includes a connector assembly with tubing to connect to a cooling unit. FIG. 15 illustrates the system including the cooling unit 1509. FIG. 14B shows a side view of the patient wearing the applicator 1501 shown in FIG. 14A.

In this example, the applicator is compact, including a skin-contacting thermal surface (cooling surface 1505) that is cooled by a thermal control fluid that is chilled and pumped into the applicator from the cooling unit. The applicator may be held against the skin by an adhesive 1503. An optional border may include the adhesive, and in addition, adhesive may be on the cooling surface as well. In some variations the adhesive is a material that aids in thermal transfer (e.g., a hydrogel). The applicator also includes one or more sensors 1517 that may be adjacent or on the cooling transfer surface.

As mentioned above, any appropriate sensor may be used, including sensors to detect heart rate (HR and/or HRV), skin temperature, blood pressure, skin conductivity/impedance, etc. In some variations the sensor may be connected to the controlling unit via the connector assembly 1507 that may also include the fluid lines for a thermal transfer fluid.

The applicator, and particularly the skin-contacting thermal surface 1505 may be small. For example, the largest diameter of the skin-contacting thermal surface may be 10 cm or less (e.g., 9 cm or less, 8 cm or less, 6 cm or less, 7 cm or less, 5 cm or less, 4 cm or less, etc., e.g., between 2 and 10 cm, between 2 and 8 cm, between 4 and 8 cm, etc.).

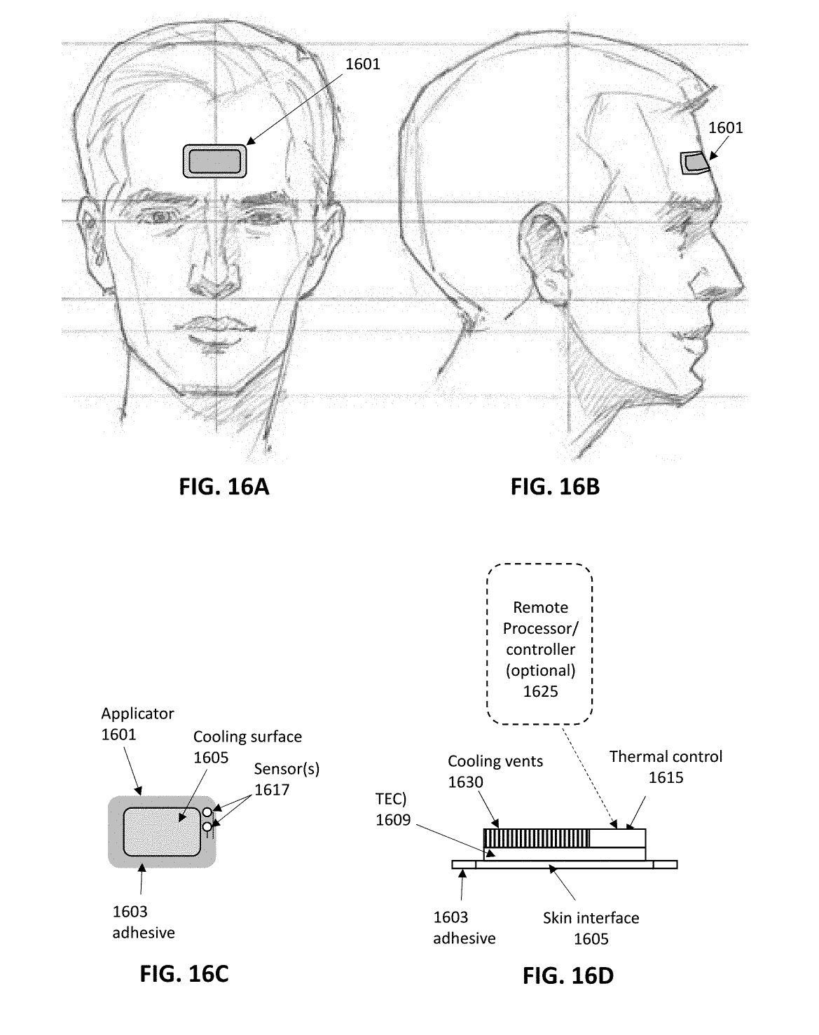

The cooling unit 1509 in FIG. 15 is separate from the applicator; in other variations (as shown in FIGS. 16A-16B, below), the cooling unit may be integrated with the applicator and worn on the patient's face. In general, the cooling unit may include one or more thermal control modules 1515 that may include a thermoelectric cooler (TEC), fans, heat skins, heat distributors, etc. In variations in which a thermal transfer fluid is used, the thermal control module may include a reservoir of fluid (e.g., a tank and/or a cartridge that may be refillable), pump and fluid passages for cooling and moving thermal transfer fluid to and from the applicator via the connector assembly. In the example shown in FIG. 15, a cartridge 1511 is included that may provide thermal transfer fluid to the thermal control. The cartridge may be removable and/or refillable and/or replaceable.

The cooling unit may also include a controller 1517 that includes control logic. The controller may include control circuitry, memory, one or more processors, and the like for storing and executing control logic to maintain the temperature of the applicator (e.g., the skin-contacting thermal surface of the applicator). The controller may also include or be functionally connected to sensor circuitry and/or logic to process data from the one or more sensors. In variations in which feedback from the one or more sensors is used, the sensor data or a processed version of the sensor data may be used by the controller to control the temperature. For example, in variations in which the sensor senses heart rate, the controller may receive HR data, or raw data including HR information and may extract the HR data (such as HRV or high-frequency HRV). This HR data may then be used to set the temperature of the applicator. For example, in variations in which HRV is used as feedback, when the HRV is within a predetermined distance (e.g., 2%, 5%, 7%, 9%, 10%, 12%, 15%, 17%, 20%, 25%, etc.) of the baseline HRV, cooling may be applied (e.g., cooling to a maximum amount within a range (e.g., within 5-15 degrees, 10-15 degrees, etc.); when the HRV is outside of this range (e.g., greater than the predetermined distance) relative to the baseline, the temperature may be increased within the defined range (e.g., closer to 15 degrees in variations in which 15 degrees C. is the upper range limit).

Any of these cooling units may also include an optional user interface which may allow display of one or more parameters, and indication of the status of the device and/or an on/off control. In some variations a control may be provided to allow the user to set the temperature of the apparatus. For example, the user may be provided with a control to allow the user to adjust the temperature up or down by some amount (e.g., +/−1 degree, 2 degrees, 3 degrees, etc. relative to the current value).

In some variations the control unit is configured to wirelessly communicate with a remote processor or controller 1525. For example, the apparatus may include wireless communication circuitry 1521 allowing wireless (e.g., Wi-Fi, Bluetooth, ZigBee, etc.) communication. The wireless communication circuitry may be functionally connected and/or part of the controller.

FIG. 16A-16B illustrate another variation of an apparatus for selectively modulating the trigeminal nerve by cooling that is attached to the skin of the patient's forehead at the midline. In this example, the apparatus is self-contained, and does not include a separate cooling unit, but many of the components of the cooling unit shown in FIG. 15 are integrated into the applicator 1601. FIGS. 16C and 16D illustrate the apparatus with a skin-contacting thermal surface (skin interface 1605) and an adhesive 1603 similar to that described in FIG. 14A and 15. The cooling unit may be attached to the back of the applicator, and may include a TEC 1609, cooling vents 1630 and the controller 1615; any of the features included in FIG. 15 and discussed above may be included in this variation as well, with the exception of the connector assembly to the extent that it is not necessary.

The patient-facing surface of the application, including the cooling surface 1605 (which may have the same dimensions discussed above), sensor(s) 1617, and adhesive 1603 may be included. The apparatus may also include wireless communication circuitry and may wirelessly communicate with a remote processor 1625, such as a smartphone, tablet, computer, etc. In some variations a user interface may be displayed on the remote device and may remotely control the apparatus. For example, an smartphone may display an app (e.g., application software) that receives and sends information with the apparatus to control operation of the apparatus, receive and store data (e.g., compliance data, sensor data, operational data, operational errors, etc.) and may transmit control information (on/off, temperature setting, dosing time/duration, etc.).

Any of these apparatuses may also include a power source (e.g., battery) and control circuitry controlling the power to the apparatus.

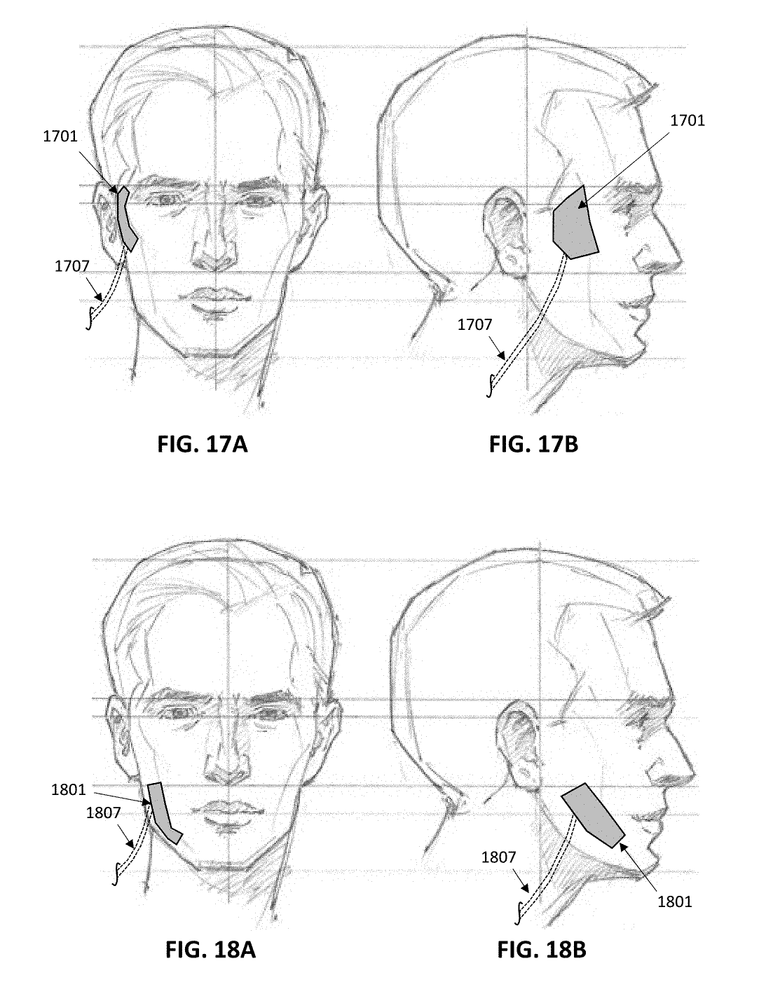

FIGS. 17A-17B and 18A-18B illustrate alternative apparatuses configured for placement to other regions of the patient's face. For example, FIGS. 17A and 17B illustrate an apparatus configured to be worn over the maxillary never, e.g., between the ear and the eye on one side of the patient's face. In this example the apparatus 1701 may provide cooling via an integrated (as in FIGS. 16A-16B) or remote cooling unit (as in FIG. 15) though a connector assembly 1707. In FIG. 17A-17B, the applicator may include a crescent-shaped skin-contacting thermal surface that is cooled; the crescent or loosely C-shaped (e.g., convex) surface may provide good contact with the skin over the maxillary nerve.

In FIG. 18A-18B, the apparatus 1801 is configured to attach to the side of the patient's jaw. In FIGS. 18A and 18B, for example, the apparatus may be bent or curved to conform to the outer perimeter of the patient s jaw, for placement over the mandibular nerve. In variations in which a remote cooling unit and fluid is used to cool the device (e.g., FIG. 15) a connecting assembly 1807 may be used.

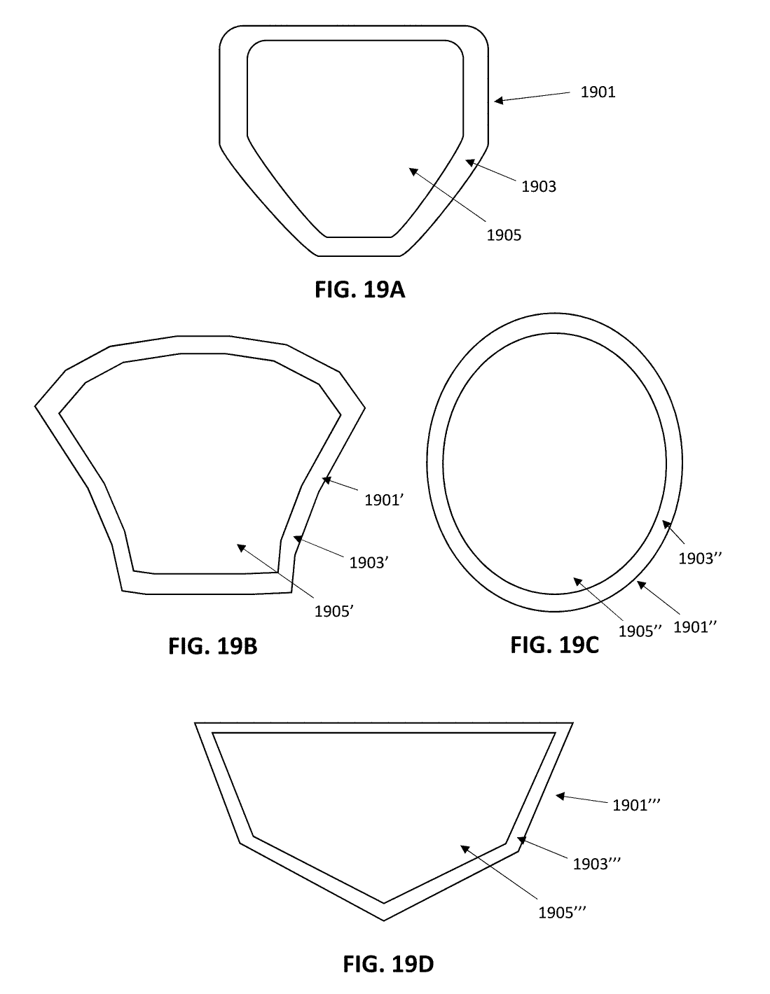

FIGS. 19A-19D illustrate other variations of the skin-contacting thermal surface 1905, 1905′, 1905″, 1905″′ that may be used as part of an apparatus 1901, 1901′, 1901″, 1901″′. In these examples a border of adhesive 1903, 1903′, 1903″, 1903′ is included, but may be optional; as mentioned, in some variation the apparatus may be held to the patient without an adhesive (e.g., via a strap, band, belt, etc.) and/or the adhesive may be on the skin-contacting thermal surface. Applicators configured to be worn on the forehead may be tapered, e.g., having a larger upper or top region compared to the lower region when worn on the face (the lower region is worn closer to the nose), as shown in FIGS. 19A, 19B and 19D. In some variations the skin-contacting thermal surface from which the thermal transfer (cooling) occurs is smaller than the overall region worn on the head. For example, other regions may be covered (and in some variations thermally insulated), but not cooled.

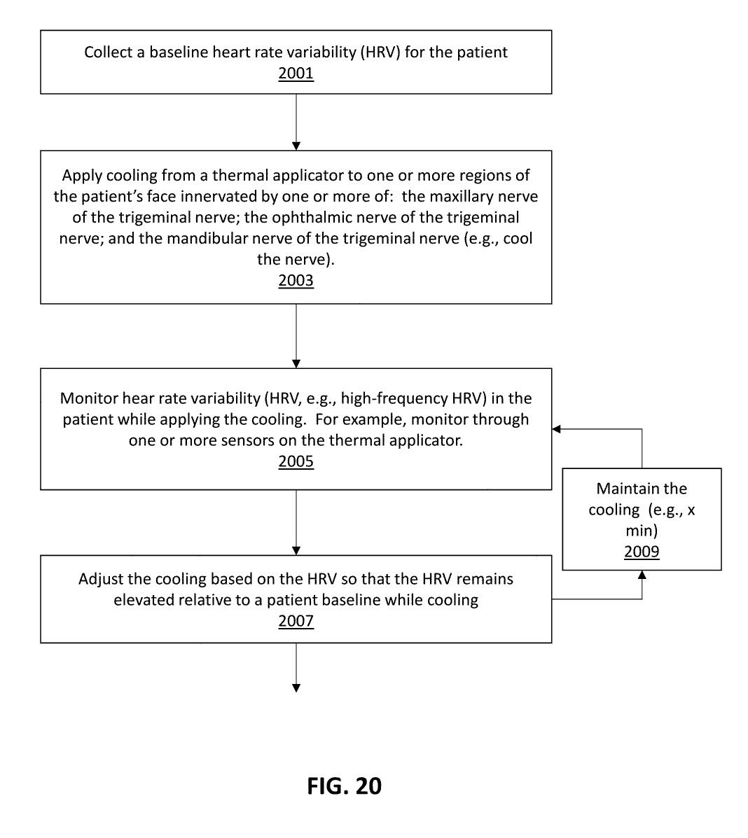

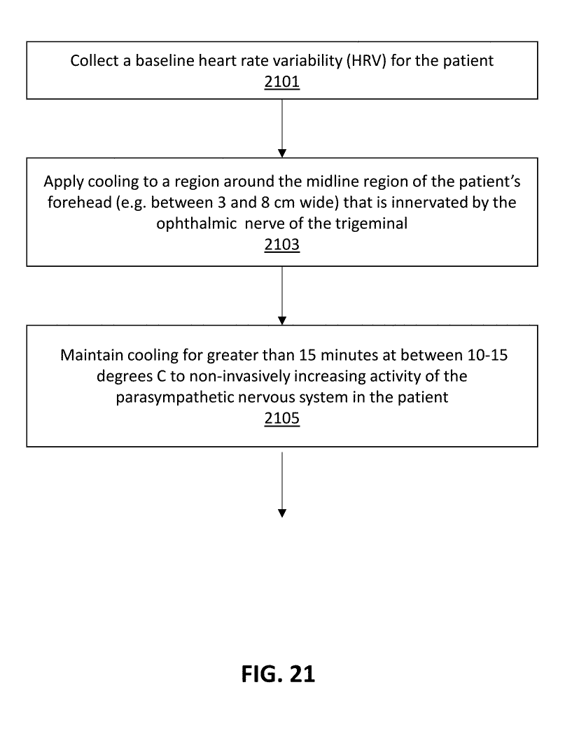

FIGS. 20 and 21 illustrate exemplary methods of treatment of a patient. For example, FIG. 20 schematically illustrates one method of treating of non-invasively increasing activity of the parasympathetic nervous system in a patient. In this example, a baseline for the parasympathetic feedback variable (e.g., HR, HRV, etc.) is first determined. This may be empirically measured and averaged from the patient. In some variations the baseline may be constrained to within a reasonable range to prevent unreasonable baselines. The baseline may be measured for a predetermined amount of time (e.g., 1 minute, 2 minutes, 5 minutes, etc.) immediately before applying the cooling. For example, in FIG. 20, a baseline is collected for HRV 2001. Once collected, cooling may be applied. Prior to collecting the baseline, the apparatus may be applied to the patient's face in the desired location.

In this example, for example, cooling may be applied from the thermal applicator to a region of the patient's face that is innervated by one or more of: the maxillary nerve of the trigeminal nerve; the ophthalmic nerve of the trigeminal nerve; and the mandibular nerve of the trigeminal nerve 2003. To apply cooling, the controller may set the apparatus to a target temperature and control the TEC or other cooling components to the target temperature. Temperature may be initially ramped to the target temperature.

The patient may be monitored (e.g., for the HRV or other feedback variable) while applying the cooling through one or more sensors on the thermal applicator 2005. The controller may adjust the temperature by controlling the cooling based on feedback. For example, the apparatus may adjust the cooling based on the HRV so that the HRV remains elevated relative to a patient baseline while cooling 2007. This process may be iterative and feedback may begin once the device has reached the initial target temperature (thus any of these apparatus may include a temperature sensor, such as a thermistor sensing the temperature to be found at the skin-contacting thermal surface). For example, during the maintenance of the temperature, which may be sustained for treatment time (e.g., 5 minutes, 10 minutes, 15 minutes, 20 minutes, 25 minutes, 30 minutes, etc.) 2009, the patient may be monitored and the temperature adjusted.

FIG. 21 shows a similar variation in which the method is a method is used for applying cooling to the trigeminal to induce a diving reflex using an applicator having a small skin-contacting thermal surface. In this example, the applicator is placed on the skin (e.g., over the forehead, on the jaw, between the eye and ear, etc.) and a baseline for the feedback variable is taken 2101. Once the baseline is collected and/or adjusted, cooling to an initial target temperature (e.g., between 10-15 degrees C.) is begun 2103. Once the target temperature is reached, it may be maintained for a treatment time 2105, e.g., greater than 15 minutes. In some variations, the temperature may be adjusted and/or the treatment time may be adjusted based on the feedback variable (e.g., HRV). For example, the patient may be treated by cooling until the time for an elevated HRV is sustained (on aggregate) for greater than a minimum (e.g., 15 minutes or more).

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

We claim:

1. A method of non-invasively increasing activity of the parasympathetic nervous system in a patient, the method comprising:
applying cooling from a thermal applicator to a region of the patient's face that is innervated by the trigeminal nerve;
monitoring heart rate variability (HRV) in the patient while applying the cooling; and
adjusting the cooling based on the HRV so that the HRV remains elevated relative to a patient baseline while cooling; and
maintaining the cooling for at least 15 minutes.

2. The method of claim 1, wherein applying comprises applying to a region of the forehead along the midline of the forehead.

3. The method of claim 1, wherein applying comprises applying to a region of the midline of the forehead having a diameter of less than 6 cm.

4. The method of claim 1, wherein applying comprises applying to a region innervated by the maxillary nerve (V₂) of the trigeminal nerve.

5. The method of claim 1, wherein applying comprises applying to a region innervated by the ophthalmic nerve (V₁) of the trigeminal nerve.

6. The method of claim 1, wherein applying comprises applying to a region innervated by the mandibular never (V₃) of the trigeminal nerve.

7. The method of claim 1, wherein applying comprises applying from an applicator adhesively attached to the patient's face.

8. The method of claim 1, wherein monitoring HRV comprises monitoring high-frequency HRV.

9. The method of claim 1, wherein monitoring HRV comprises monitoring HRV from a sensor on the thermal applicator.

10. The method of claim 1, further comprising gathering a baseline HRV from the patient before applying cooling.

11. The method of claim 1, wherein adjusting the cooling based on the HRV comprises lowering the temperature of the thermal applicator if the HRV is not elevated above a predetermined threshold compared to the patient baseline while cooling.

12. The method of claim 1, wherein adjusting the cooling based on the HRV comprises adjusting the cooling between about 10-15 degrees C.

13. The method of claim 1, wherein maintaining the cooling for at least 15 minutes comprises maintaining the cooling for at least 30 minutes.

14. The method of claim 1, further comprising repeating the steps of applying, monitoring, adjusting and maintaining at least twice a day for 10 days.

15. The method of claim 1, wherein maintaining the cooling for at least 15 minutes comprises treating the patient for a neurological disorder by maintaining the cooling for at least 15 minutes, wherein the neurological disorder is one or more of: depression, attention deficit hyperactivity disorder (ADHD), epilepsy, and migraines.

16. A method of non-invasively increasing activity of the parasympathetic nervous system in a patient, the method comprising:
collecting a baseline heart rate variability (HRV) for the patient;
applying cooling from a thermal applicator to a region of the patient's face that is innervated by one or more of: the maxillary nerve of the trigeminal nerve; the ophthalmic nerve of the trigeminal nerve; and the mandibular nerve of the trigeminal nerve;
monitoring HRV in the patient while applying the cooling through one or more sensors on the thermal applicator; and
adjusting the cooling based on the HRV so that the HRV remains elevated relative to a patient baseline while cooling; and
maintaining the cooling for at least 15 minutes.

17. A method of selectively modulating a patient's trigeminal nerve by regionally cooling the trigeminal nerve, the method comprising:
applying cooling to a 6 cm wide region around the midline region of the patient's forehead that is innervated by the ophthalmic nerve of the trigeminal nerve;
maintaining cooling for greater than 15 minutes at between 10-15 degrees C. to non-invasively increasing activity of the parasympathetic nervous system in the patient.

18. The method of claim 17, wherein applying comprises applying from an applicator adhesively attached to the patient's face.

19. The method of claim 17, further comprising monitoring heart rate variability (HRV).

20. The method of claim 17, further comprising monitoring high-frequency heart rate variability (HRV).

21. The method of claim 17, further comprising monitoring heart rate variability (HRV) comprises monitoring HRV from a sensor on a thermal applicator attached to the patient's forehead.

22. The method of claim 17, further comprising gathering a baseline heart rate variability (HRV) from the patient before applying cooling.

23. The method of claim 17, further comprising adjusting the cooling based on the heart rate variability (HRV) by lowering the temperature of a thermal applicator on the patient's forehead if the HRV is not elevated above a predetermined threshold compared to the patient baseline while cooling.

24. The method of claim 23, wherein adjusting the cooling based on the HRV comprises adjusting the cooling between about 10-15 degrees C.

25. The method of claim 17, wherein maintaining the cooling for at least 15 minutes comprises maintaining the cooling for at least 30 minutes.

26. The method of claim 17, further comprising repeating the steps of applying and maintaining at least twice a day for 10 days.

27. The method of claim 17, wherein maintaining the cooling for at least 15 minutes comprises treating the patient for a neurological disorder by maintaining the cooling for at least 15 minutes, wherein the neurological disorder is one or more of: depression, attention deficit hyperactivity disorder (ADHD), epilepsy, and migraines.

28. An apparatus for selectively modulating the trigeminal nerve by cooling, the apparatus comprising:
an applicator having a skin-contacting thermal surface, wherein the skin-contacting thermal surface is 6 cm or less in diameter;
one or more sensors adjacent to or within the skin-contacting thermal surface, wherein the one or more sensors are configured to sense heart rate;
an adhesive adjacent to the skin-contacting thermal surface of the thermal applicator to secure the skin-contacting thermal surface to a patient's skin; and
a thermal control configured to control the temperature of the skin-contacting thermal surface at between about 10-15 degrees C.

29. The apparatus of claim 28, further comprising a connector assembly to a cooling unit, wherein the thermal control is contained at least partially within the cooling unit.

30. The apparatus of claim 29, further comprising a user interface on the cooling unit, wherein the user interface is configured to allow the user to control the application of cooling.

31. The apparatus of claim 28, further comprising a fluid cartridge configured to deliver cooled, temperature-controlled fluid to the cooling surface of the applicator.

32. An apparatus for non-invasively increasing activity of the parasympathetic nervous system in a patient, the apparatus comprising:
an applicator having a skin-contacting thermal surface that is configured to attach to a region of the patient's face that is innervated by the patient's trigeminal nerve to apply cooling;
one or more heart rate (HR) sensors on the applicator, the one or more HR sensors configured to measure the patient's heart rate;
control circuitry configured to control the temperature of the skin-contacting thermal surface between 10-15 degrees C. and further configured to monitor heart rate variability (HRV) in the patient while applying the cooling from the applicator, wherein the control circuitry is further configured to adjust the temperature of the skin-contacting thermal surface based on the patient's HRV.

33. The apparatus of claim 32, wherein the control circuitry is configured to adjust the temperature of the skin-contacting thermal surface based on the patient's HRV so that the HRV remains elevated relative to a patient baseline while cooling the skin-contacting thermal surface.

34. The apparatus of claim 32, further wherein the skin-contacting thermal surface is 6 cm or less in diameter.

35. The apparatus of claim 32, wherein the one or more heart rate sensors are adjacent to or within the skin-contacting thermal surface.

36. The apparatus of claim 32, further comprising an adhesive adjacent to the skin-contacting thermal surface of the thermal applicator to secure the skin-contacting thermal surface to a patient's skin.

37. The apparatus of claim 32, wherein the skin-contacting thermal surface is tapered at a bottom and wider at the top.

38. The apparatus of claim 32, further comprising a connector assembly connecting the applicator to a cooling unit housing the control circuitry.

39. The apparatus of claim 38, further comprising a user interface on the cooling unit, wherein the user interface is configured to allow the user to control the application of cooling.

40. The apparatus of claim 32, further comprising a fluid configured to circulate through a channel in thermal communication with the skin-contacting thermal surface, wherein the temperature of the fluid is controlled by the control circuitry.

41. The apparatus of claim 32, further comprising a thermoelectric cooler (TEC) in thermal communication with the skin-contacting thermal surface.

42. The apparatus of claim 41, wherein the TEC is positioned adjacent to the skin-contacting thermal surface on the applicator.

43. The apparatus of claim 32, wherein the skin-contacting thermal surface of the applicator has a substantially crescent shape and the applicator is configured to adhesively attach to the side of the patient's face between the patient's eye and the patient's ear to cool the patient's maxillary nerve.

44. The apparatus of claim 32, wherein the skin-contacting thermal surface of the applicator has an angled shape, having an angle of between 100 and 135 degrees and the applicator is configured to adhesively attach to the side of the patient's jaw to cool the patient's mandibular nerve.
